(19) United States
(12) Patent Application Publication
(10) Pub. No.: US 2021/0128635 A1
(43) Pub. Date: May 6, 2021

(54) Composition for Regenerating Hepatic Tissue

(71) Applicant: The Nippon Dental University, Tokyo (JP)

(72) Inventors: Ken Yaegaki, Tokyo (JP); Hiroshi Ishikawa, Tokyo (JP)

(21) Appl. No.: 16/641,285

(22) PCT Filed: Aug. 25, 2017
(86) PCT No.: PCT/JP2017/030496

§ 371 Date: Jan. 15, 2021

(51) Int. Cl. A61K 35/32; A61P 1/16; A61K 9/00

(52) U.S. Cl. CPC A61K 35/32; A61K 9/0019; A61P 1/16

(57) ABSTRACT

Provided is a composition for liver tissue regeneration, in particular, a composition for liver tissue regeneration for prevention or treatment of a liver disease. The composition for liver tissue regeneration includes a human dental pulp stem cell as an effective component.

### TECHNICAL FIELD

The present invention relates to a composition for liver tissue regeneration, in particular, a composition for liver tissue regeneration for prevention or treatment of a liver disease.

### BACKGROUND ART

Conventionally, cell transplantation has been carried out for the purpose of repairing or regenerating a tissue, or for treating a disease. Cell transplantation can be expected to have a direct therapeutic effect since cells themselves constituting the tissue, or cells that support construction of the tissue, are administered to an adult. For example, in the fields of liver tissue regeneration and treatment of liver diseases, preclinical studies and clinical studies have so far been carried out by transplantation of, for example, CD34-positive bone marrow stem cells to the liver or the like (for example, Non-patent Document 1). However, from the viewpoint of treatment of a liver disease, such transplantation of CD34-positive bone marrow stem cells only allows slight improvement of the liver disease, and never cures the disease. Thus, the effect of the transplantation has merely been prolonging of the survival time, or the period that can be spent for waiting for cadaveric liver transplantation or living donor liver transplantation.

On the other hand, since dental pulp stem cells can be easily collected from deciduous teeth and extracted teeth which have conventionally been discarded, such as milk teeth and wisdom teeth, those stem cells are expected to be useful for tissue generation, or for prevention or treatment of a disease. However, there has been no established method which allows differentiation/maturation of liver cells from dental pulp stem cells, to thereby enabling preparation (regeneration) of a large amount of liver tissue in the body of a mammal. Moreover, at present, there is no established method for preventing or treating a liver disease using dental pulp stem cells.

### PRIOR ART DOCUMENT

### Non-Patent Document

Non-patent Document 1: Nakamura et al., Journal of Gastroenterology and Hepatology, Vol. 29, 2014, pages 1830-1838

### SUMMARY OF THE INVENTION

Recently, the present inventors intensively studied to discover that a liver tissue can be effectively regenerated using particular dental pulp stem cells. The present invention is based on such a discovery.

Accordingly, an object of the present invention is to provide a composition for liver tissue regeneration in mammals, the composition comprising a human dental pulp stem cell as an effective component.

More specifically, examples of the present invention include the following.
[1] A composition for liver tissue regeneration in a mammal, the composition comprising a human dental pulp stem cell as an effective component.
[2] The composition for liver tissue regeneration according to [1], wherein the human dental pulp stem cell is differentiated into a hepatocyte.
[3] The composition for liver tissue regeneration according to [1] or [2], wherein the human dental pulp stem cell is a human deciduous dental pulp stem cell.
[4] The composition for liver tissue regeneration according to any one of [1] to [3], wherein the human dental pulp stem cell expresses CD29, CD73, CD90, CD105, and CD166, but does not express CD34, CD45, and CD133.
[5] The composition for liver tissue regeneration according to any one of [1] to [4], for prevention or treatment of a human liver disease.
[6] The composition for liver tissue regeneration according to any one of [1] to [4], for prevention or treatment of a liver disease of a non-human mammal.
[7] The composition for liver tissue regeneration according to [5] or [6], wherein 5×10⁶ to 4×10¹⁰ human dental pulp stem cells per 15 kg body weight of the human or non-human mammal, whose liver disease is to be prevented or treated, are administered to the human or non-human mammal.
[8] The composition for liver tissue regeneration according to any one of [1] to [7], to be administered to the splenic sinus of the human or non-human mammal.
[9] The composition for liver tissue regeneration according to any one of [5] to [8], wherein the liver disease is selected from the group consisting of liver cancer, liver failure, liver cirrhosis, hepatitis, non-alcoholic fatty liver disease, and metastatic cancer, and a disease state requiring adjunctive therapy for a side effect caused by cancer chemotherapy.
[10] A prophylactic or therapeutic method for a liver disease in a human or non-human mammal, the method comprising administering an effective amount of human dental pulp stem cells to the human or non-human mammal.
[11] The method according to [10], wherein the human dental pulp stem cells are administered to the splenic sinus of the human or non-human mammal.
[12] The method according to [10] or [11], wherein the liver disease is selected from the group consisting of liver cancer, liver failure, liver cirrhosis, hepatitis, non-alcoholic fatty liver disease, and metastatic cancer, and a disease state requiring adjunctive therapy for a side effect caused by cancer chemotherapy.

According to the present invention, liver tissue generation of mammals can be carried out using human dental pulp stem cells, which can be easily collected. Further, according to the present invention, prevention or treatment of liver diseases such as non-alcoholic fatty liver disease (NAFLD) can be effectively carried out.

### BRIEF DESCRIPTION OF THE DRAWINGS

FIG. 1 is a biopsy photograph of a liver tissue of a liver disease (non-alcoholic fatty liver disease (NAFLD)) model animal (micromini pig).

FIG. 2 is a graph illustrating the blood albumin levels before the application, at the beginning of the application, and at Week 4 after the beginning of the application, of the composition of the present invention.

FIG. 3 is a graph illustrating the total blood cholesterol levels before the application, at the beginning of the application, and at Week 4 after the beginning of the application, of the composition of the present invention.

FIG. 4 is a graph illustrating the blood type IV collagen levels before the application, at the beginning of the application, and at Week 4 after the beginning of the application, of the composition of the present invention.

FIG. 5 is a graph illustrating the blood hyaluronic acid levels before the application, at the beginning of the application, and at Week 4 after the beginning of the application, of the composition of the present invention.

FIG. 6 is a graph illustrating the blood alanine aminotransferase (ALT) levels before the application, at the beginning of the application, and at Week 4 after the beginning of the application, of the composition of the present invention.

FIG. 7 is a biopsy photograph of a spleen tissue of a liver disease model animal (micromini pig) at Week 4 after the application of the composition of the present invention to the spleen.

### MODE FOR CARRYING OUT THE INVENTION

The composition for liver tissue regeneration of the present invention comprises a human dental pulp stem cell as an effective component. The human dental pulp stem cell contained in the composition of the present invention may be derived from any of a human milk tooth, permanent tooth, and wisdom tooth. The human dental pulp stem cell is preferably a dental pulp stem cell derived from a deciduous milk tooth or wisdom tooth.

The dental pulp stem cell used in the present invention may be preliminarily prepared before carrying out the present invention, or may be a dental pulp stem cell which is generally commercially available. In cases where the dental pulp stem cell is prepared, the individual from which the dental pulp stem cell is collected may be either the same or different from the individual to which the composition containing the dental pulp stem cell is to be applied. However, from the viewpoint of suppressing occurrence of rejection due to application of the composition, and enhancing the graft survival efficiency and regeneration efficiency, the dental pulp stem cell is preferably collected from the same individual. Examples of the human dental pulp stem cell which is generally commercially available include human dental pulp stem cells (manufactured by CLI; product number, 300702-SF) and hDPSC-dental pulp stem cells (manufactured by Lonza Japan; product number, PT-5025).

In the case where preliminarily prepared dental pulp stem cells are used in the present invention, the dental pulp stem cells may be collected and treated by, for example, the following method. In this collection-treatment method, (1) collection of dental pulp, (2) enzyme treatment, (3) cell culture, and (4) collection of cells, are carried out in this order.

### (1) Collection of Dental Pulp

A human milk tooth extracted immediately before it naturally drops, or a permanent tooth such as a wisdom tooth, is disinfected with a chlorhexidine or Isodine solution, and a dental pulp tissue is collected therefrom through the root canal using a sterilized broach.

### (2) Enzyme Treatment

The dental pulp tissue collected is treated with 4 mg/ml collagenase I and dispase at 37° C. for 1 hour.

### (3) Cell Culture

A cell suspension obtained by the enzyme treatment is plated on a dish having an area of 25 cm², and cultured at a CO₂ concentration of 5% in Dulbecco's modified Eagle medium (DMEM) supplemented with 10% fetal bovine serum (FBS), 100 U/ml penicillin, 100 μg/ml streptomycin, and 0.25 μg/ml amphotericin B. The cells are cultured to 85 to 90% confluence, and then plated on a fresh dish having an area of 25 cm² at about 10⁵ cells/cm².

### (4) Collection of Cells

The cultured cells are detached from the dish by trypsin treatment or the like, and then centrifuged to collect the cells, followed by resuspending the cells in Hanks' balanced salt solution (HBSS) at about 2×10⁶ cells/100 μl. The thus collected cells are used as dental pulp stem cells to be used in the present invention.

Examples of a method for confirming the fact that the prepared cells are dental pulp stem cells include a method in which the expression pattern(s) of a marker(s) specific to dental pulp stem cells is/are used as an index/indices. More specifically, one of the characteristics of dental pulp stem cells is that they express CD29, CD73, CD90, CD105, and CD166, but that they do not express CD34, CD45, and CD133. Expression of each of these markers can be detected and confirmed by immunofluorescent staining, FACS (fluorescence activated cell sorting), or the like.

The human dental pulp stem cells used in the present invention are preferably used after allowing their differentiation into hepatocytes. Examples of the method of differentiation of dental pulp stem cells into hepatocytes include, but are not limited to, subjecting dental pulp stem cells to a conventionally known method (such as the method described in Okada et al., “Hydrogen sulphide increases hepatic differentiation of human tooth pulp stem cells compared with human bone marrow stem cells,” International Endodontic Journal). More specifically, dental pulp stem cells are cultured to about 70% confluence in serum-free DMEM supplemented with 1% insulin-transferrin-sodium selenite and embryotrophic factor, and then the cells are treated for 5 days by addition of 20 ng/ml hepatocyte growth factor (HGF), followed by treatment for 15 days by addition of oncostatin M and dexamethasone.

The method of confirming the differentiation of the dental pulp stem cells into the hepatocytes is not limited, and examples of the method include a method in which the resulting cells are practically transplanted to the liver of a living body to confirm their differentiation capacity, and a method in which the expression level(s) of a gene marker(s) characteristic to differentiated hepatocytes, such as albumin and/or a-fetoprotein, is/are analyzed by ELISA, real-time RT-PCR, or the like.

The human dental pulp stem cells contained in the composition of the present invention may be dental pulp stem cells in the separated state. However, from the viewpoint of enhancing the graft survival efficiency and regeneration efficiency of the dental pulp stem cells after the application of the composition, dental pulp stem cells forming spheres may be used. The composition of the present invention may be either in the solid form or in the liquid form as long as the composition contains an effective amount of human dental pulp stem cells. Depending on the method of application of the composition, the composition may be prepared as a solid or liquid composition containing a pharmaceutically acceptable carrier or additive.

The amount of human dental pulp stem cells contained in the composition of the present invention is not limited as long as a liver tissue can be regenerated therewith. The amount may be appropriately set depending on the type of the mammal to which the composition is applied, and on the weight of the mammal. For example, 5×10⁶ to 4×10¹⁰ dental pulp stem cells may be contained per 15 kg body weight of the mammal to which the composition is applied. The composition preferably contains 1×10⁷ to 4×10⁷ dental pulp stem cells per 15 kg body weight of the subject mammal.

The subject to which the composition of the present invention is to be applied is not limited as long as it is a mammal. The subject may be a human, or may be a non-human mammal. The composition of the present invention is especially preferably used for, for example, a higher mammal belonging to Primates, Artiodactyla, Cetartiodactyla, Perissodactyla, or the like.

The site of application of the composition of the present invention is not limited, and may be an organ such as the liver, spleen, or lung. Regarding the method of application of the composition of the present invention, the composition is applied to the site of application such as an organ, a tissue, or the like by a conventionally known method such as injection, implantation, packing, or coating. In cases where the composition of the present invention is prepared using an appropriate carrier such as a solvent into the form of a gel having an appropriate fluidity, application of the composition is possible by a simple method such as packing, injection, or coating. Further, in cases where the composition is in the form of a gel, it can be simply applied to the target organ or the like using an injection needle or the like. For example, in cases where the composition of the present invention is applied to the spleen, it may be directly applied to the spleen, or may be applied by injection into the splenic sinus using a syringe to allow transfer of the composition into the spleen through the bloodstream. It has been confirmed that, when the composition of the present invention is used to regenerate a liver tissue in an organ other than the liver, the regeneration of the liver tissue is possible without deteriorating the function of the organ.

When a liver tissue is regenerated using the composition of the present invention, the animal to which the composition is to be applied is preferably subjected to immunosuppressive treatment before the application of the composition, so as to suppress immune reaction caused by the application of the composition. The immunosuppressive treatment may be carried out by using a commercially available immunosuppressive agent. Examples of the commercially available immunosuppressive agent include tacrolimus (product name: Prograf (registered trademark); manufactured by Astellas Pharma Inc.). The immunosuppressive treatment may be carried out by a conventionally known method suitable for the immunosuppressive agent employed.

In cases where the composition of the present invention is applied using Prograf as an immunosuppressive agent, Prograf and the composition of the present invention may be applied according to, for example, the following schedule.

First, on the day before the application of the composition of the present invention, both in the early morning and at night, a 1-mg package(s) (granules, 500 mg) and/or 0.2-mg package(s) (granules, 100 mg) of Prograf granules is/are used for oral administration of 0.075 mg/kg body weight of the granules (total daily dose, 0.15 mg/kg body weight). On the day of the application, an aqueous solution of Prograf in physiological saline, prepared by dissolving Prograf injection (5 mg/ml) in 200 ml of physiological saline at 0.15 mg/kg body weight, is intravenously administered by continuous infusion of 100 ml of the solution for 2 hours both in the early morning and at night (total daily dose, 0.15 mg/kg body weight). From the next day after the application to Day 28 after the application, an aqueous solution of Prograf in physiological saline, prepared by dissolving Prograf injection (5 mg/ml) in 200 ml of physiological saline at 0.10 mg/kg body weight, is intravenously administered by continuous infusion of 100 ml of the solution for 2 hours both in the early morning and at night (total daily dose, 0.10 mg/kg body weight). When the composition of the present invention is administered to an individual having a body weight of 15 kg, 1125 mg of Prograf (two 1-mg packages and one and a quarter 0.2-mg packages of Prograf granules) is orally administered both in the morning and in the evening on the day before the application of the composition. On the day of the application, an aqueous solution of Prograf in physiological saline, prepared by dissolving 2.25 mg of Prograf (450 μl of Prograf injection (5 mg/ml)) in 200 ml of physiological saline, is intravenously administered by continuous infusion of 100 ml of the solution for 2 hours both in the early morning and at night. From the next day after the application to Day 28 after the application, an aqueous solution of Prograf in physiological saline, prepared by dissolving 1.5 mg of Prograf (300 μl of Prograf injection (5 mg/ml)) in 200 ml of physiological saline, is intravenously administered by continuous infusion of 100 ml of the solution for 2 hours both in the early morning and at night. Preferably, from the next day after the application of the composition of the present invention, the blood level of Prograf is measured every day, and an aqueous solution of Prograf in physiological saline, prepared by dissolving 1.5 mg of Prograf (300 μ of Prograf injection (5 mg/ml)) in 200 ml of physiological saline, is intravenously administered as appropriate such that the blood level is kept higher than 0.01 mg/ml.

Four to ten weeks after the application of the composition of the present invention, a tissue (cell population) containing: human dental pulp stem cells contained in the composition of the present invention applied; and cells derived therefrom by division or growth; is collected. Whether or not regeneration of a liver tissue has occurred is confirmed by comparing the collected tissue (cell population) with a liver tissue of a normal individual by histological analysis such as histological staining; immunostaining against liver markers; in situ hybridization; ELISA; analysis using RT-PCR or real-time RT-PCR; electron microscopy; or the like. Examples of the liver markers include, but are not limited to, albumin, a-fetoprotein, CPS (carbamoyl-phosphate synthetase), and mitochondria. In cases where the expression levels of these liver markers are almost the same as those in the liver of the normal individual, or in cases where these liver markers are applied to a subject having a deficiency in the function of the liver, and the deficiency in the function of the liver is improved and/or complemented, the cell population collected can be judged to be a differentiated liver tissue, that is, a regenerated liver tissue.

The composition of the present invention can be used for prevention or treatment of a mammalian liver disease. Thus, according to another mode of the present invention, a pharmaceutical composition comprising the composition of the present invention is provided. According to still another mode of the present invention, use of the composition of the present invention in production of a pharmaceutical composition is provided. According to a preferred mode of the present invention, the pharmaceutical composition is used for prevention or treatment of a mammalian liver disease. In the pharmaceutical composition, the composition of the present invention may be used as it is, or after adding a pharmaceutically acceptable carrier as appropriate thereto.

The liver disease to be prevented or treated with the composition of the present invention is not limited, and examples of the liver disease include liver cancer, liver failure, liver cirrhosis, hepatitis, non-alcoholic fatty liver disease, and metastatic cancer, and a disease state requiring adjunctive therapy for a side effect caused by cancer chemotherapy. More specifically, the disease state requiring adjunctive therapy for a side effect caused by cancer chemotherapy means a disease state such as liver failure or metastatic liver cancer. In the mammal to which the composition of the present invention is to be applied, the liver disease may be complicated with a lifestyle-related disease such as obesity, diabetes, hyperlipidemia, or hypertension.

A prophylactic or therapeutic effect of the composition of the present invention on a liver disease may be confirmed, after application of the composition of the present invention, based on pathological findings obtained by histological staining of the whole organ or tissue to which the composition of the present invention was applied, and/or based on an ordinary blood test. Here, “the whole organ or tissue to which the composition of the present invention was applied” means an organ or a tissue containing a cell population containing: human dental pulp stem cells contained in the composition of the present invention applied; and cells derived therefrom by division or growth.

In cases where a prophylactic or therapeutic effect of the composition of the present invention on a liver disease is confirmed based on a blood test, the blood test is carried out by comparing at least one parameter selected from the following (1) to (6) with that of normal individuals.
(1) Total blood cholesterol level
(2) Blood albumin level
(3) Hepaplastin test (HPT) value
(4) Blood type IV collagen level
(5) Blood alanine aminotransferase (ALT) level
(6) Blood hyaluronic acid level

More specifically, after application of the composition of the present invention to a mammalian individual with a liver disease, if the at least one parameter shows almost the same value as those in normal individuals, or if the at least one parameter tends to show a value closer to those in normal individuals, the composition of the present invention is judged to have a prophylactic or therapeutic effect on the liver disease.

According to another mode of the present invention, a method of preventing or treating the liver disease in a mammal, the method comprising administering the composition of the present invention to the mammal, is provided. In the method, the composition of the present invention is administered such that human dental pulp stem cells in an amount effective for preventing or treating the liver disease in the mammal are administered to the mammal. The method includes not only a mode in which a composition of the present invention comprising human dental pulp stem cells is administered, but also a mode in which human dental pulp stem cells are administered as they are.

The effective amount of the human dental pulp stem cells is not limited, and is appropriately determined by a physician depending on the type, age, sex, conditions, and/or the like of the subject mammal.

The method of administering the human dental pulp stem cells, or the composition of the present invention comprising the human dental pulp stem cells, is not limited. It/they may be administered according to, for example, the same procedure as the above-described procedure for regeneration of a liver tissue.

### EXAMPLES

The present invention is described below in more detail by way of Examples. However, the present invention is not limited by these Examples.

### Preparation of Dental Pulp Stem Cells

According to a conventional method, milk teeth were collected from healthy humans of 6 to 12 years old. The collection of the milk teeth, and preparation and use of dental pulp stem cells from the collected milk teeth, were carried out after obtaining informed consent from each subject or his/her parent. From each collected milk tooth, a dental pulp tissue was collected through the root canal using a sterilized broach. The dental pulp tissue collected was digested using 4 mg/ml dispase II (manufactured by GIBCO; product number, 1728484) and 4 mg/ml collagenase I (manufactured by Wako Pure Chemical Industries, Ltd.; product number, CTH2297) at 37° C. for 1 hour. The cell suspension after the digestion was subjected to culture on a dish (manufactured by TPP) having an area of 25 cm² using Dulbecco's modified Eagle medium (DMEM) (manufactured by Invitrogen; supplemented with 100 U/ml penicillin, 100 μg/ml streptomycin, and 0.25 μg/ml amphotericin B) supplemented with 10% fetal bovine serum (FBS), in an atmosphere with a CO₂ concentration of 5% at 37° C. The cells were cultured to 85 to 90% confluence, and then subcultured on a fresh dish at 1×10⁵ cells/cm².

After three times of subculture in DMEM medium supplemented with 10% FBS, cells were detached from the dish using trypsin. The detached cells were subjected to the Manual MACS (registered trademark) Cell Separation protocol to isolate CD117⁺ dental pulp stem cells. The CD117⁺ dental pulp stem cells were cultured for 5 days in serum-free DMEM supplemented with 20 ng/ml hepatocyte growth factor (manufactured by HGF), and then 10 ng/ml oncostatin M (manufactured by R&D) and 10 nM dexamethasone (manufactured by Wako Pure Chemical Industries, Ltd.) were added to the medium, followed by performing culture for additional 15 days. The cells after the culture were suspended in Hanks' balanced salt solution (manufactured by Gibco) at about 1×10⁷ cells/ml, to provide the composition of the present invention.

### Preparation of Liver Disease Animal Model

Using micromini pigs, an animal model with a liver disease (non-alcoholic fatty liver disease (NAFLD)) was prepared by the following procedure.

About 7-month-old male micromini pigs (body weight, about 10 kg) (strain: Fuji Micromini Pig) were purchased from Fuji Micra Inc., and 12 individuals of the pigs were acclimated to a rearing environment with natural ventilation at a temperature of 20±5° C. and a relative humidity of 55±25% under a light-dark cycle of 7:00 to 19:00 (light)/19:00 to 7:00 (dark) while being fed with 300 g/day of a solid diet MP-A (manufactured by Oriental Yeast Co., Ltd.; containing 0.29% choline and 15.3% crude protein), which is a normal diet. The feeding of the pigs was carried out at about 8:00 every day.

After an acclimation period of about one week, a blood test was carried out for the 12 normal individuals. More specifically, venous blood was collected from each of the normal 12 individuals to obtain serum, and the serum sample obtained was subjected to measurement of the following items (1) to (6). The measured values were regarded as “normal values”. The items (1) to (6), and their normal values were as follows.
(1) Total blood cholesterol level: 214 mg/dl
(2) Blood albumin level: 4.192 mg/dl
(3) Hepaplastin test (HPT) value: 21.9%
(4) Blood type IV collagen level: 0.095 μg/ml
(5) Blood alanine aminotransferase (ALT) level: 32 U/I
(6) Blood hyaluronic acid level: 32 ng/ml

Each of the items (1) to (6) was measured using a method, kit, or the like described below.
(1) The total blood cholesterol level was measured by the cholesterol dehydrogenase (UV) method.
(2) The blood albumin level was measured by nephelometry (the modified BCP method).
(3) The hepaplastin test (HPT) value was measured by coagulation time measurement.
(4) The blood type IV collagen level was measured using a Type IV collagen ELISA kit, ACB (manufactured by Funakoshi Corporation).
(5) The blood alanine aminotransferase (ALT) level was measured by the JSCC reference method.
(6) The blood hyaluronic acid level was measured by the latex agglutination turbidimetric immunoassay.

After the measurement of the normal values, the pigs were fed ad libitum with 300 g/day of CDAA diet (manufactured by Research Diets, Inc.; product number, A15022101), which is substantially free of protein, in the same rearing environment for 16 weeks. The feeding of the pigs was carried out at about 8:00 every day. The 12 individuals after the 16 weeks of ad libitum feeding were used as a liver disease model. The 12 individuals of the liver disease model were subjected to a blood test in the same manner as the normal individuals.

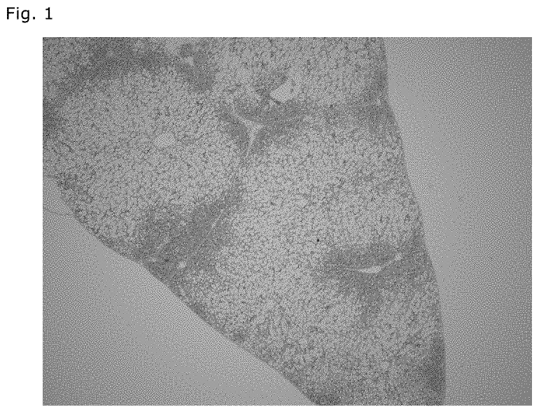

From one individual out of the liver disease model individuals obtained, a liver tissue was removed, and a biopsy photograph was obtained therefrom. The biopsy photograph is shown in FIG. 1. In the biopsy photograph shown in FIG. 1, the gray areas correspond to hepatocytes, and the white small granular areas correspond to lipid (lipid droplets). The micromini-pig liver tissue obtained by the method of the present invention showed deposition of a large amount of lipid (lipid droplets) in hepatocytes, which is not found in normal liver tissues. Thus, a liver disease was found also from the biopsy photograph.

The results of the blood test for the 12 individuals of the liver disease model were as follows. These measured values were used as “disease values”.
(1) Total blood cholesterol level: 87 mg/dl
(2) Blood albumin level: 1.768 mg/dl
(3) Hepaplastin test (HPT) value: 6.9%
(4) Blood type IV collagen level: 0.998 μg/ml
(5) Blood alanine aminotransferase (ALT) level: 246 U/I
(6) Blood hyaluronic acid level: 51 ng/ml

In the results of the blood test on the liver disease findings (markers), (1) a decrease in the total blood cholesterol level, (2) a decrease in the blood albumin level, (3) a decrease in the HPT value, (4) an increase in the blood type IV collagen level, (5) an increase in the blood ALT level, and (6) an increase in the blood hyaluronic acid level, were found.

Thus, based on the findings from the biopsy photograph and the blood test, the liver disease model obtained was found to be a liver disease model which can be extrapolated to the liver disease (NAFLD).

### <Example: Study of Therapeutic Effect of Application of Composition Containing Dental Pulp Stem Cells to Liver Disease>

A composition of the present invention containing dental pulp stem cells, prepared by the above-described method, was applied to the spleen of the liver disease model prepared by the above-described method. By this, the effect of the composition of the present invention on treatment of the liver disease was studied.

The 12 individuals of the liver disease model prepared by the above-described method were divided into an application group, which is composed of 6 individuals to which the composition of the present invention is applied, and a control group, which is composed of 6 individuals to which the composition is not applied.

In the application group, the composition of the present invention containing dental pulp stem cells was applied to the spleen of the liver disease model by the following method. First, on the day before the beginning of application of the composition of the present invention, tacrolimus (product name: Prograf (registered trademark) Granules; manufactured by Astellas Pharma Inc.) was orally administered to the six individuals in the application group both in the early morning and at night at a dose of Prograf of 0.075 mg/kg body weight per administration (total daily dose, 0.15 mg/kg body weight), to cause immunosuppression. Subsequently, the composition of the present invention prepared by the above-described method (containing about 1×10⁷ dental pulp stem cells/ml) was injected into the splenic sinus of each of the six individuals in the application group using a 1-ml syringe (manufactured by Terumo Corporation; product number: SS-01T). Four weeks after the beginning of the application, venous blood was collected from each individual to obtain serum, and the serum sample obtained was subjected to a blood test. On the day of application of the composition of the present invention, both in the early morning and at night, Prograf (registered trademark) injection (5 mg/ml) was dissolved in 100 ml of physiological saline (manufactured by Otsuka Pharmaceutical Co., Ltd.; product number, D05352) such that the dose of Prograf per administration is 0.075 mg/kg body weight, and the resulting solution was intravenously administered to cause immunosuppression (total daily dose, 0.15 mg/kg body weight). From the next day after the application of the composition of the present invention, both in the early morning and at night, Prograf (registered trademark) injection (5 mg/ml) was dissolved in 100 ml of physiological saline such that the dose of Prograf per administration is 0.05 mg/kg body weight, and the resulting solution was intravenously administered to cause immunosuppression (total daily dose, 0.10 mg/kg body weight). From the next day after the application of the composition of the present invention, the blood level of Prograf in each individual was measured every day, and an aqueous solution of Prograf in physiological saline, prepared by dissolving 1.5 mg of Prograf (300 μl of Prograf injection (5 mg/ml)) in 200 ml of physiological saline, was intravenously administered as appropriate such that the Prograf level in venous blood was about 0.013 mg/ml, to maintain the immunosuppression. In the control group, the same volume of physiological saline was administered instead of Prograf.

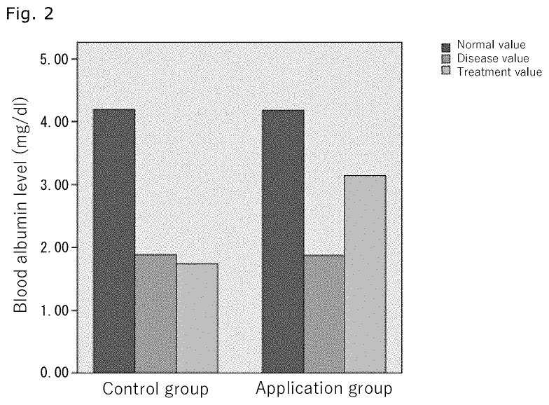

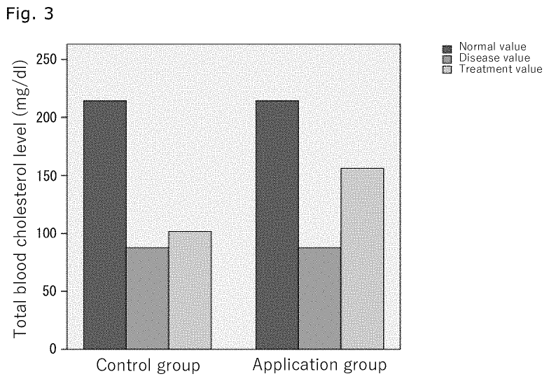

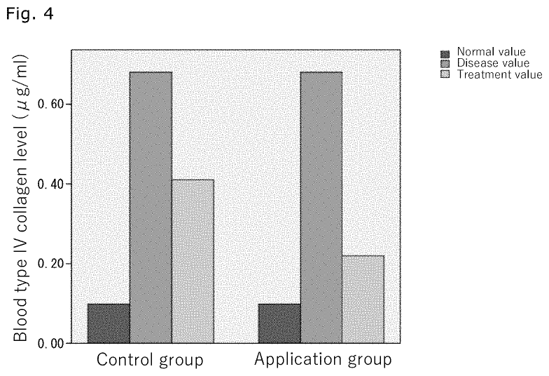

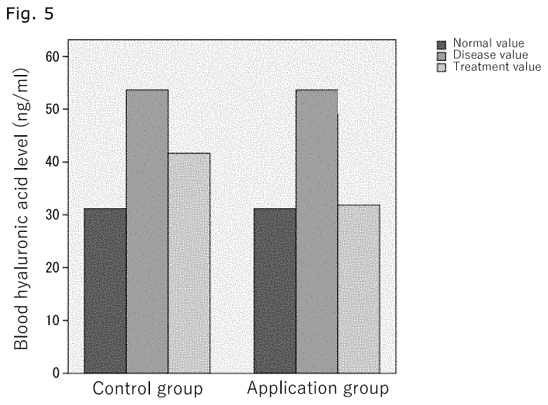

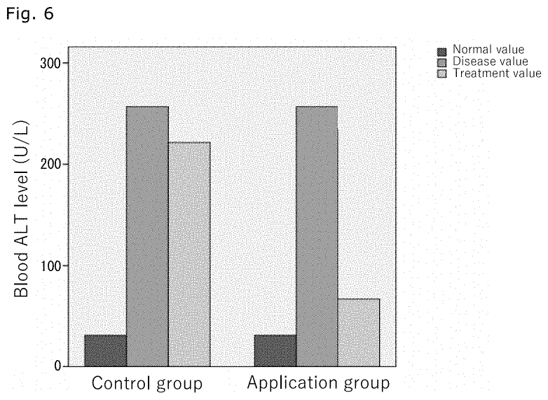

FIGS. 2 to 6 are graphs corresponding to the respective items, each summarizing the result of the blood test (the average for the six individuals, “treatment value”), the normal value, and the disease value obtained for each of the application group and the control group. As is evident from the results in FIGS. 2 to 6, at Week 4 after the beginning of the application of the composition of the present invention, the liver disease markers in the application group showed values closer to their normal values in the blood test (changes from the disease values to the treatment values). This means that the liver disease was cured by the application of the composition of the present invention. Although not shown in the figures, in the blood test carried out at Week 4 after the beginning of the application of the composition of the present invention, the hepaplastin test (HPT) value was 11.0%, and the blood type IV collagen level was 0.382 μg/ml. Since the values of these liver disease markers at Week 4 after the beginning of the application of the composition of the present invention also tended to be closer to the normal values, it is suggested that the liver disease was cured by the application of the composition of the present invention.

On the other hand, in the control group, the liver disease markers hardly showed differences between the disease values and the treatment values. More specifically, each treatment value tended to be slightly closer to its normal value, or was more distant from the normal value than the disease value was. These results indicate that the liver disease was hardly cured, or that the liver disease rather progressed.

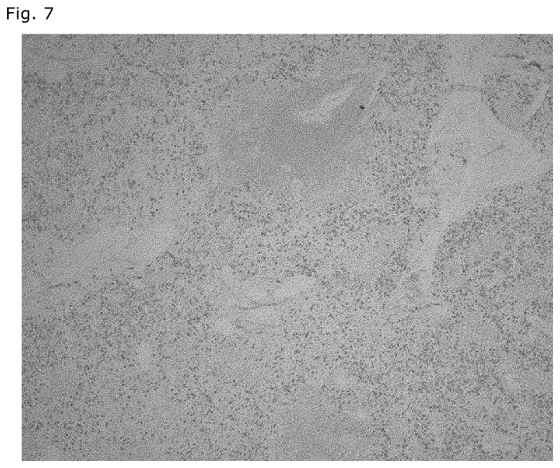

From one individual in the application group, a spleen tissue was removed at Week 4 after the beginning of the application of the composition of the present invention, and the tissue was stained with diaminobenzidine (DAB). A biopsy photograph obtained after the staining is shown in FIG. 7. In the biopsy photograph shown in FIG. 7, the black granular areas correspond to hepatocytes. It can be seen, from this biopsy photograph, that regeneration of a liver tissue occurred in the spleen to which the composition of the present invention was applied, indicating that, so to speak, a second liver complementing the function of the liver was created. According to pathological findings, the biopsy photograph obtained as a result shows a pathological image indicating that regeneration of the liver tissue further proceeds with time, leading to curing of the liver disease.

Thus, based on the blood test and the biopsy photograph, it became clear that application of the composition of the present invention containing human dental pulp stem cells effectively allows regeneration of a liver tissue, and prevention or treatment of a liver disease based on the regeneration. Further, it can be said, from this result, that the composition of the present invention containing human dental pulp stem cells can be used for regeneration of a liver tissue and for prevention or treatment of a liver disease.

### INDUSTRIAL APPLICABILITY

By the present invention, a composition for regeneration of a liver tissue and for prevention or treatment of a liver disease can be produced using human dental pulp stem cells which can be easily collected from deciduous teeth and extracted teeth.

1. A composition for liver tissue regeneration in a mammal, the composition comprising a human dental pulp stem cell as an effective component.

2. The composition for liver tissue regeneration according to claim 1, wherein the human dental pulp stem cell is differentiated into a hepatocyte.

3. The composition for liver tissue regeneration according to claim 1, wherein the human dental pulp stem cell is a human deciduous dental pulp stem cell.

4. The composition for liver tissue regeneration according to claim 1, wherein the human dental pulp stem cell expresses CD29, CD73, CD90, CD105, and CD166, but does not express CD34, CD45, and CD133.

5. The composition for liver tissue regeneration according to claim 1, for prevention or treatment of a human liver disease.

6. The composition for liver tissue regeneration according to claim 1, for prevention or treatment of a liver disease of a non-human mammal.

7. The composition for liver tissue regeneration according to claim 5, wherein 5×10⁶ to 4×10¹⁰ human dental pulp stem cells per 15 kg body weight of the human or non human mammal, whose liver disease is to be prevented or treated, are administered to the human or non human mammal.

8. The composition for liver tissue regeneration according to claim 1, to be administered to the splenic sinus of a human or non-human mammal.

9. The composition for liver tissue regeneration according to claim 1, wherein the liver disease is selected from the group consisting of liver cancer, liver failure, liver cirrhosis, hepatitis, non-alcoholic fatty liver disease, and metastatic cancer, and a disease state requiring adjunctive therapy for a side effect caused by cancer chemotherapy.

10. A prophylactic or therapeutic method for a liver disease in a human or non-human mammal, the method comprising administering an effective amount of human dental pulp stem cells to the human or non-human mammal.

11. The method according to claim 10, wherein the human dental pulp stem cells are administered to the splenic sinus of the human or non-human mammal.

12. The method according to claim 10, wherein the liver disease is selected from the group consisting of liver cancer, liver failure, liver cirrhosis, hepatitis, non-alcoholic fatty liver disease, and metastatic cancer, and a disease state requiring adjunctive therapy for a side effect caused by cancer chemotherapy.

13. The composition for liver tissue regeneration according to claim 6, wherein 5×10⁶ to 4×10¹⁰ human dental pulp stem cells per 15 kg body weight of the non-human mammal, whose liver disease is to be prevented or treated, are administered to the non-human mammal.
